(19) United States
(12) Patent Application Publication
(10) Pub. No.: US 2020/0147328 A1
(43) Pub. Date: May 14, 2020

(54) ELECTROMECHANICAL BREATH ACTUATED INHALER

(71) Applicant: CIPLA LIMITED, Mumbai (IN)

(72) Inventors: Nandan KULKARNI, Pune, Maharashtra (IN); Vidur MALHOTRA, Mumbai, Maharashtra (IN); Geena MALHOTRA, Mumbai, Maharashtra (IN); Brinder AHLUWALIA, Bangalore, Karnataka (IN); Mohnish Jagdish ZAVERI, Mumbai, Maharashtra (IN)

(21) Appl. No.: 16/630,383

(22) PCT Filed: Jul. 11, 2018
(86) PCT No.: PCT/IN2018/050453

§ 371 Date: Jan. 10, 2020

(30) Foreign Application Priority Data
Jul. 11, 2017 (IN) 201721024441

(51) Int. Cl. A61M 15/00

(52) U.S. Cl. CPC A61M 15/0091; A61M 2205/3553; A61M 2205/52; A61M 2205/3334

(57) ABSTRACT

A breath-actuated inhaler includes a motion-generator configured to render motion upon actuation by a user. An arrangement transforms the generated motion to enable a canister at undergoing translation-motion. A metering-valve is operably connected to the canister to cause a metered-release of the canister's contents as a spray, based upon the translation-motion.

### FIELD OF THE INVENTION

The present disclosure relates to metered dose inhalers (MDI) and in particular relates to assembly and functionality of the MDI.

### BACKGROUND AND PRIOR ART

Currently known devices for delivering aerosol medications for inhalation by patient include metered dose inhaler that are manually operated and breath-actuated. The metering dose inhaler body contains a receptacle for the canister and a valve actuator. A valve-stem from the canister is terminated by a nozzle, wherein a spray created by the nozzle is directed outside the receptacle through a mouthpiece, which acts as a protrusion of the receptacle. The valve stem is typically passive. Accordingly, when the canister is pressed against the receptacle, by manual or automatic advance, the valve-stem is pressed within the metering-valve and releases the dose via the flow path out of the nozzle.

Breath actuated devices provide a metered dose automatically when patients' inspiratory flow rate rises above a preset threshold. Typically, mechanical breath actuated devices such as Autohaler®, Easy-Breathe contain one or more mechanical means of actuation by using stored energy in the spring component to actuate the canister. These devices deploy a mechanical component such as flap, lever, housing, spring-bellow etc to detect the threshold flow rate reached and accordingly trigger the actuator-component. Few examples of conventionally existing inhalers may be depicted as follows:

U.S. Pat. No. 5,347,998 describes a breath-actuated inhaler (electromechanical) with electromechanical priming mechanism, wherein the electromechanical assembly is involved only to trigger the dose but not in the activation of the trigger point itself. This device does priming action electromechanically to relieve patient from doing it manually. Further the inhaler provides an electromechanical means to relieve the firing load imposed on the aerosol canister during the actuation.

U.S. Pat. No. 5,284,133 describes a dose timer, actuator mechanism and a patient compliance monitoring means. The invention relates to a dose or timing controlled actuator that operates in conjunction with an inhalation device to prevent both patient under-compliance with prescribed medication and patient abuse of or dependence on prescribed medication.

U.S. Pat. No. 5,497,764 describes a portable, battery powered, hand-held system for releasing a controlled dose of aerosol medication for inhalation by a patient including a durable body and an aerosol medication cassette inserted in the durable body. Actuator mechanism includes a compression spring for depressing the canister and a torsion spring for reloading the compression spring. Torsion spring is reloaded by rotating the cassette from an open position for delivering aerosol to a closed position.

U.S. Pat. No. 5,404,871 describes an apparatus and method for delivering an amount of aerosolized medicine for inspiration by a patient in response to the occurrence of an appropriate delivery point or points in the patient's breath flow. Changes in breath flow pattern during the course of an aerosolized medication inspiration therapy program may be detected and used to adjust the controlled amount of medication to be delivered in a given administration and/or to conform to the pattern of the patient's condition or change in condition.

At least a problem as persisting in the use of metered dose inhalers is that the user often actuates the metered dose inhaler and then begins inhalation. Such an inhalation/medicating pattern limits an amount of medicament delivered to the lung and causes most of the medicament to impact the mouth and throat. Accordingly, the user obtains less than optimal-dose of medicament.

Existing breath-actuated inhalers are not designed to accommodate available aerosol canisters separate from the receiving bodies or housings for which they were originally marketed and approved by regulators such as (FDA). Since the housing greatly influences the nature of the aerosol spray-characteristics as well as the amount of medication received by the patient, the separate accommodation of the canister within the housing causes the canister to be non-rigidly disposed, thereby adversely affecting the spray-characteristics.

Another problem with many of mechanical breath activated inhalers is that the aerosol canister remains in the depressed-position until the patient physically intervenes and relieves the mechanical load on the canister by moving a lever, strap, button, guides or some other mechanical means. Immediately after the venting, the metering chamber of the canister becomes vulnerable to the intrusion of air and the extent of air intrusion increases with the length of time canister is in “depressed” position. This may result in vapor-locking in the metering chamber, and hence incomplete filling of the chamber of the valve, thereby rendering a risk of incomplete dosage

Another problem with mechanical breath actuated devices is that aerosol canister must be in the ‘ready-to-fire’ or armed position to allow recovery of aerosol canister from depressed position under action of its own internal valve spring. This has two implications potentially—a) mechanism may be armed during the intervals between the inhaler use, and b) actuator may be armed during the storage which is particularly of concern as the functional-life and reliability of the device may be compromised by the long-term stress effects of maintaining the actuation mechanism in armed position.

Typically, the actuation mechanism contains element of spring or mechanical energy restoring based element, which may creep or relax upon prolonged storage and may cause premature-firing or delayed/extended firing time. Such spring-components as used in the construction of known mechanical or electromechanical breath actuated devices generally remain in a compressed position and often cause injury and pose safety issues, in case the device gets accidently activated completely due to either shock or in the post-usage disposal handling.

Another problem with electromechanically actuated inhalers is requirement of a large battery size to drive the components. Moreover, the inhalers typically lack in terms of operating in real-time to customize the actuation-length, trigger time as per patient-inhalation/patient-breathing profile and generally don't provide a direct bidirectional-communication with the patient. This renders these devices as a minor variant of standalone breath-actuated devices which lack a feature to provide the patients a feedback or gather feedback from patients based on dose tracking, adherence monitoring and coordination control. Overall, majority of the electronic-inhalers merely end up providing posterior information as to timing of the dose taken, correctness of inhalation maneuvers, etc.

### SUMMARY OF THE INVENTION

This summary is provided to introduce a selection of concepts in a simplified format that are further described in the detailed description of the present disclosure. This summary is neither intended to identify key or essential inventive concepts of the disclosure, nor is it intended for determining the scope of the invention or disclosure.

The present subject matter at least refers to a breath-actuated inhaler comprising a motion-generator configured to render motion upon actuation by a user. An arrangement is provided for transforming the generated motion to enable the canister undergo translation-motion, and a metering-valve is operably connected to the canister for causing a metered-release of the canister's contents as a spray, based upon said translation-motion.

In another embodiment, the present subject matter at-least refers to a method implemented in a breath-actuated inhaler. The method comprises converting a rotary motion from a motion generator into translation-motion upon a user-actuation to thereby depress a canister. Thereafter, at least one of following is monitored:

a) an inhalation-flow generated due to said user-actuation, through a pressure-sensor;

b) motion as underwent by the motion generator and the canister, through a motion-sensor; and

c) an instantaneous position exhibited by the motion-generator and canister, through a position sensor.

The method further comprises processing data obtained based on said monitoring to execute one or more of:

a) control of the timing of actuation of the canister;

b) control of the timing of an auto return mechanism of the canister;

c) control of the time of venting of the metered dose inhaler.

d) real-time tracking of the inhalation flow-rate;

e) real-time control of amount of medication for inhalation;

f) derivation of an inhalation-profile of the user;

g) judgment of inhalation done by the user;

h) acknowledgement of a complete-dosage as undertaken by the patient;

i) maintaining a count of dosages in a given period of time;

j) sending a real-time feedback regarding an executed inhalation; and

k) communication of data pertaining to operation of the inhaler to a remote-device.

In an embodiment, the present subject matter describes an electronic breath-actuated inhaler comprises:
an upper-housing accommodating one or more of:
   the motion generator; and
   the canister removably connected to the motion generator to undergo translation motion;
   and
a lower-housing removably connected to said upper-housing and receiving the portion of canister comprising the metering-valve, said lower-housing comprising:
   a nozzle rigidly disposed within the lower-housing and connected to the metering valve for causing a spray formation; and
   a flow-director within the lower-housing to direct said spray outside the lower-housing;

In an implementation of said embodiment, the motion-generator in the upper housing comprises:
an electronically controllable rotary motion-source provided with a position sensor; and
an arrangement to convert the rotary motion into translation-motion, wherein said arrangement comprises:
   a lead-screw having external threads connected to said rotary-motion source; and
   a nut having internal threads coupled to said lead-screw and adapted to undergo translation-motion with the rotation of the lead-screw along a screw-length.

In an implementation of said embodiment, the motion-source in the upper housing comprises:
an electronically controllable rotary motion-source provided with a position sensor; and
a cam arrangement to convert the rotary motion into translation-motion.

In an implementation of said embodiment, the nut is rigidly connected to the lead-screw through a cap, said cap recessed from opposite-sides to accommodate said screw-nut and a portion of said canister from opposite sides.

In an implementation of said embodiment, the breath actuated inhaler comprises:
a position-sensor to sense the position of at least one of the motion-generator and the canister;
a motion-sensor to determine extent of motion underwent by motion-generator and the canister; and
a pressure-sensor provided within a flow-path of the spray to monitor an inhalation-flow as generated corresponding to the spray.

In an implementation, the microprocessor is adapted to process the received parameters to cause execution of one or more of:
control of the timing of actuation of canister;
control of the timing of auto return mechanism of canister;
control of the time of venting of the metered dose inhaler.
real-time tracking of the inhalation flow rate;
real-time control of amount of medication for inhalation;
deriving an inhalation-profile of the user;
confirmation of a complete inhalation as executed by the user;
confirmation of a complete dosage as undertaken by the patient;
logging a count of dosages in a given period of time; and
sending a real-time feedback regarding an executed inhalation.

In an implementation, the microprocessor is further adapted to trigger wireless-communication of data pertaining to the aforesaid executed operations

In an implementation, the upper housing comprising the motion source is detachable from the lower housing to enable a manual actuation of the canister without the assistance of the motion source and screw-nut.

As per the present subject-matter, the real-time data gathered during the actuation is used to process the information and actively control a trigger-timing during the inhalation maneuvers as well as control the time of inhalation to maximize coordination between actuation and inhalation, wherein the triggering is customized in accordance with the profile of the user during each dose taken.

Further, the present subject matter does not rely upon any spring-element, has substantially low components in actuation-assembly, primarily rests in very low power consumption mode, thereby eliminating the lifecycle-malfunctioning risks. This is substantially advantageous against existing mechanical or electromechanical breath actuated devices, which deploy at least one spring component in the pre-compressed mode for priming and actuation mechanism, and retain the valve stem in depressed position. Accordingly, the present-invention significantly does away with the drawbacks otherwise associated with creep-development in the spring element, i.e. tendency to lose the restoring-force and permanently deform in shape due to prolonged stressed condition, thereby increasing the risk of misfire or no actuation.

Furthermore, the entire assembly of actuation mechanism along with tracking, adherence monitoring and coordination control forms a compact water-tight upper housing attached to the lower housing and can be easily removed in case of emergency so that user can invoke manual intervention and use the device with manual actuation like a conventional actuator pMDI.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a sectional front view of an electronic breath-actuated inhaler, in accordance with an embodiment of the present invention.

FIG. 2 illustrates isometric-views of a complete housing of the breath actuated inhaler as depicted in FIG. 1.

FIG. 3 illustrates an exploded view of the complete housing as depicted in FIG. 2

FIG. 4 illustrates isometric-view of the inhaler of FIG. 1 without any upper-housing and an isometric view of the motion generator.

FIG. 5 illustrates a block-diagram of the processing operation and exemplary output-data as obtained by the operation of the inhaler of FIG. 1.

FIG. 6 illustrates a computing-architecture representation of the electronics in the inhaler as depicted in FIG. 1.

The elements in the drawings are illustrated for simplicity and may not have been necessarily been drawn to scale. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF FIGURES

For the purpose of promoting an understanding of the principles of the present disclosure, reference will now be made to the embodiment illustrated in the drawings and specific language will be used to describe the same. It will nevertheless be understood that no limitation of the scope of the present disclosure is thereby intended, such alterations and further modifications in the illustrated system, and such further applications of the principles of the present disclosure as illustrated therein being contemplated as would normally occur to one skilled in the art to which the present disclosure relates.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this present disclosure belongs. The system, methods, and examples provided herein are illustrative only and not intended to be limiting.

Embodiments of the present disclosure will be described below in detail with reference to the accompanying drawings.

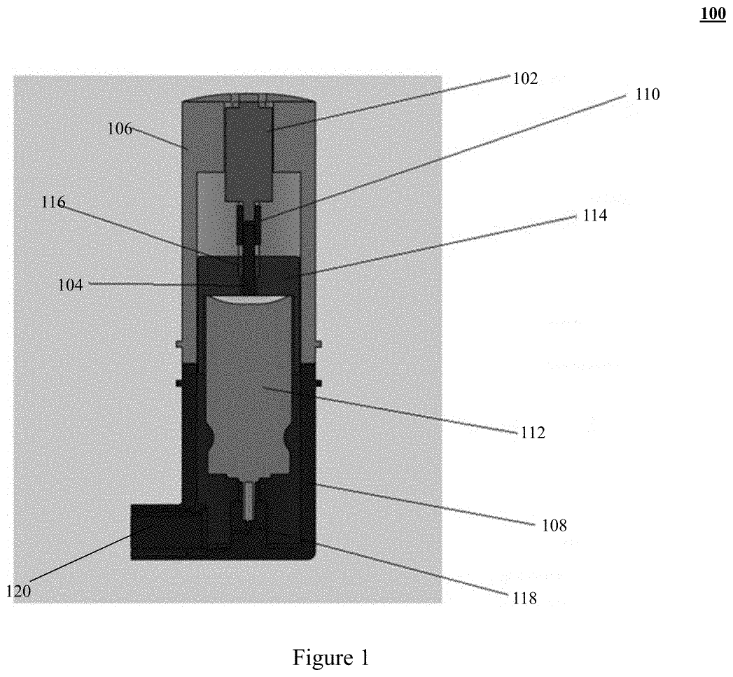

FIG. 1 illustrates a sectional front view of an Electronic breath actuated inhaler 100, in accordance with an embodiment of the present invention. Said electronic breath actuated inhalation device 100 triggers and actuates the dose using electromechanical mechanism. The electrical components/electronics within the inhaler 100 includes one or more of the components like a motion-generator 102 (e.g. a DC motor, a solenoid valve), a rechargeable-battery (not shown in figure), electronics comprising a microprocessor and a timing/triggering circuit (not shown in figure) for driving the motion-generator 102. In an example, the mechanical-components as driven by said motion generator 102 within the inhaler 100 may comprise a lead-screw 104, an off centric cam, a gear-train etc.

The aforesaid assembly of components within the inhaler 100 is constituted as a closed, water proof, disposable assembly covered by an ‘upper housing’ 106 that is removably attached to a conventional actuator body or mouthpiece-actuator of the inhaler 100. Such conventional actuator body stands referred as ‘lower housing’ 108 within the FIG. 1.

As evident from the FIG. 1, the motion generator 102 within upper housing 106 acts as an actuation mechanism and defined by the motor 102 (or DC motor). The lead-screw 104 acts as the further extension of motor 102. The DC motor 102, when supplied with electric power from a rechargeable battery unit, transfers the torque to the lead screw 104 through a viscoelastic coupling 110 and thereby enables the relative-movement of a ‘canister’ 112 with respect to the upper housing 106. Accordingly, the motion-generator 102 as an electronically-controllable rotary motion-source is configured to generate clock-wise and anti-clockwise motion for causing the translation motion of the canister 112.

As evident, the canister 112 stands partly accommodated within a cap 114 that forms a part of the upper housing 106. Such cap 114 receives the lead-screw 104 from the opposite side through an integrated nut 116, which is a recessed portion having internal-threads. Accordingly, the cap 114 undergoes translational motion as the nut 116 along the lead-screw 104 and thereby depresses the ‘canister’ 112.

More specifically, the arrangement to convert the generated motion into translation motion comprises the externally-threaded lead-screw 104 connected to the motion generator 102 and the integrated nut 116 having internal-threads coupled to the lead-screw 104 and adapted to undergo translation-motion along the screw-length of the lead-screw 104 upon the rotation of the lead-screw 104. In addition, as a part of the motion-conversion arrangement, the cap 114 is defined by a recessed top-portion acting as a screw-nut 116 (i.e. integrated-nut) to thereby translate across the length of the lead-screw 104. A lower-portion of the cap 114 is configured for holding a portion of the canister 112 to thereby enable the translation motion of the cap 114 and the canister 112 as a single unit.

Alternatively, a cam (not shown in figure) may also act as an alternate motion conversion means in-place of the lead-screw 104 and the nut 116. The cam is linked to the motion-generator 102 to convert the generated rotary-motion into translation-motion.

The inhaler 100 further comprises a nozzle 118 rigidly disposed within the lower-housing 108 and connected to a metering-valve (not show in figure) for causing a metered release of the contents of the canister 112 causing a spray-formation. Further, a flow-director 120 is provided within the lower-housing 108 of the inhaler 100 to direct said spray outside the lower-housing 108.

The inhaler 100 especially provides an automatically-controlled means to further control a timing of the auto-return mechanism of the canister 112, wherein the lead-screw return is controlled by programming a DC motor controller forming a part of the electronics within the inhaler 100.

As far as the lower housing 108 is concerned, a pressure-sensor (not shown in figure) is located in the flow path from the canister 112 to the mouthpiece detects the inhalation flow and feeds the data to the microprocessor on real time basis (say every ten milliseconds) to accurately track the inhalation flow rate.

In addition to the aforesaid pressure-sensor, the lower-housing 108 may further comprise additional pressure-sensing to check the flow-rate and provide feedback to an on-board microprocessor. Such type of utility leads to a lip-sensing arrangement in order to ascertain if the user actually inhaled through the mouthpiece and confirm the dose taken.

Overall, the microprocessor within the inhaler 100 integrates the data from pressure sensors in the flow path to detect and map an inhalation profile of the patient, identifies the triggering flow rate by self-calibrating the inhalation profile to command the actuator of the inhaler 100. Further, the microprocessor controls the duration for which a valve-stem within the inhaler 100 is discharged so as to release the medication, and to adjust the inhalation length for the dose taken.

Further, the upper housing 106 within the inhaler 100 includes a control mechanism having a position-encoder (i.e. a combination of a position and motion sensor). The control mechanism may exemplarily be a rheostat or rotational resistance-element attached to the shaft of the motor 102 for controlling the speed of the motor 102. Data from the position-encoder (i.e. which measures the position of the lead screw traversed and thereby detects the motion) and a real-time processing of the patient flow rate data from pressure-sensor enables the microprocessor to control the time of venting of the metered dose inhaler 100.

In order to further assist the computation of the microprocessor, a motion-sensor may be provided to determine extent of motion underwent by motion-generator 102 and the canister 112.

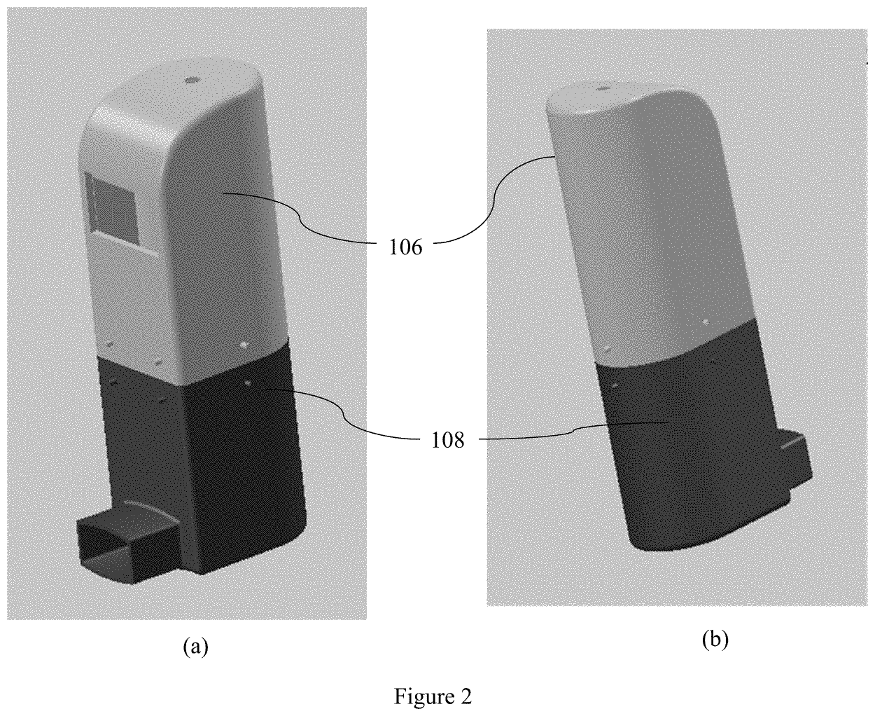

FIGS. 2(a & b) depict the schematic views of an assembly of the upper 106 and lower housing 108. The assembly constitutes a compact water-tight upper housing 106 attached to the lower housing 108, thereby making it functional under wide range of temperature, pressure operation conditions indoor as well as outdoor.

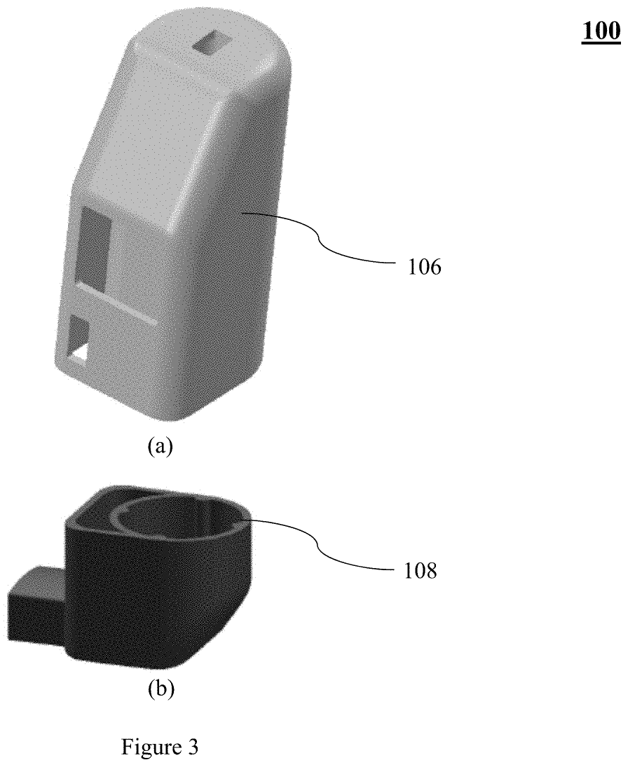

FIG. 3 depicts an exploded view with respect to FIG. 2, thereby depicting an ease of removability of the upper housing 106 from the lower housing 108. Such utility is especially advantageous in case of emergency, so that patient can invoke manual-intervention and use the device with manual actuation like a conventional pressurized metered dose inhaler (pMDI).

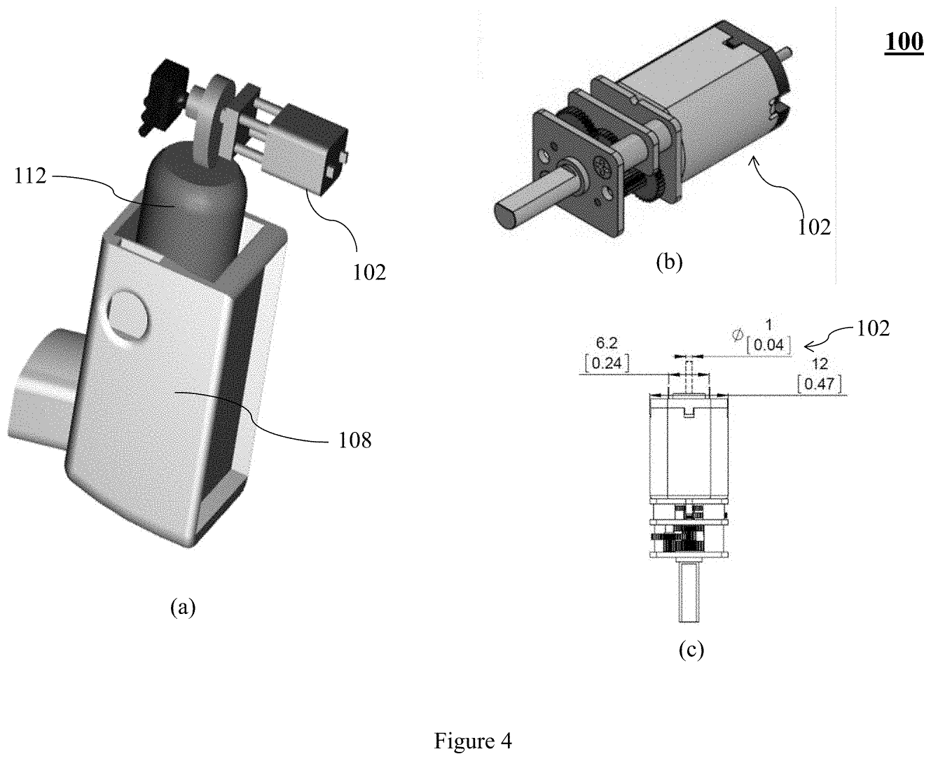

FIG. 4a depicts an isometric view of the inhaler 100 without upper housing, thereby exposing a connection between the canister 112 (as protruding from the lower housing 108) and the DC motor 102 which is interfaced to said canister 112. Further, FIG. 4b and FIG. 4c depict isometric and front views, respectively, of the DC motor 102. In addition, FIG. 4c depicts representative dimensions with respect to the DC motor 102.

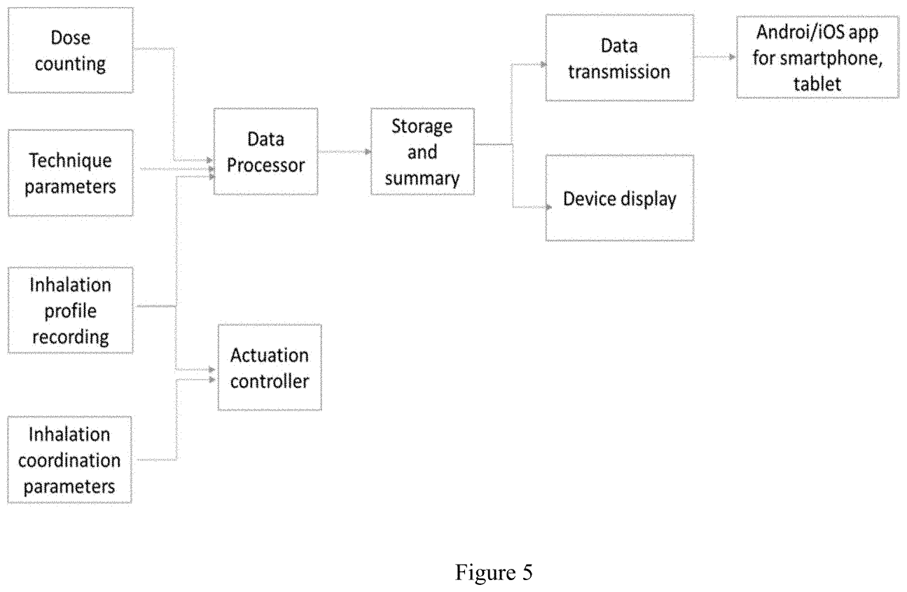

FIG. 5 illustrates a block-diagram of the processing-operation and exemplary output-data as obtained by the operation of the inhaler of FIG. 1.

The microprocessor within the inhaler 100 renders a data-logging and processing system to electronically determine at least one of:

a) at least one position parameter captured by the position sensor with respect to at least one of the motion-generator 102 and the canister 112;

b) at least one motion-parameter captured by the motion-sensor with respect to at least one of motion-generator 102 and the canister 112; and

c) a pressure-parameter associated with the spray within the flow-path, wherein the pressure-parameter is captured by at-least one pressure-sensor in respect of inhaler 100.

More specifically, the microprocessor within the present inhaler 100 electronically monitors an inhalation-flow generated due to said actuation by the pressure-sensor disposed in a flow-path; and an extent of the translation-motion as generated by the rheostat (i.e. motion and position sensor) attached to a shaft of the DC motor 102. Based on said monitoring data as obtained, the microprocessor executes one or more of the following tasks:
a) control of the timing of actuation of canister 112;
b) control of the timing of an auto return mechanism of canister 112;
c) control of the time of venting of the metered dose inhaler 100;
d) real-time tracking of the inhalation flow-rate;
e) real-time control of amount of medication for inhalation;
f) derivation of an inhalation-profile of the user;
g) judgment of inhalation done by the user;
h) acknowledgement of a complete-dosage as undertaken by the patient;
i) maintaining a count of dosages in a given period of time;
j) sending a real-time feedback regarding an executed inhalation; and
k) communication of data pertaining to operation of the inhaler to a remote-device.

Based upon data as generated pertaining to inhalation technique of user, number of dosages undertaken, historical inhalations by the user (i.e. inhalation profile), and a coordination as exhibited between the actuation and inhalation, etc, the microprocessor prepares a data summary or report for sending to the user by deploying low ‘Bluetooth’ energy based data transfer mechanism. Accordingly, the recorded data is transmitted from each actuation to an application operating upon a smartphone. The processed data also enables assistance to an ongoing usage of the inhaler 100 by communicating data as feedback to an onboard microcontroller of the DC motor 102 for causing control of inhalation length, venting time, triggering-points etc.

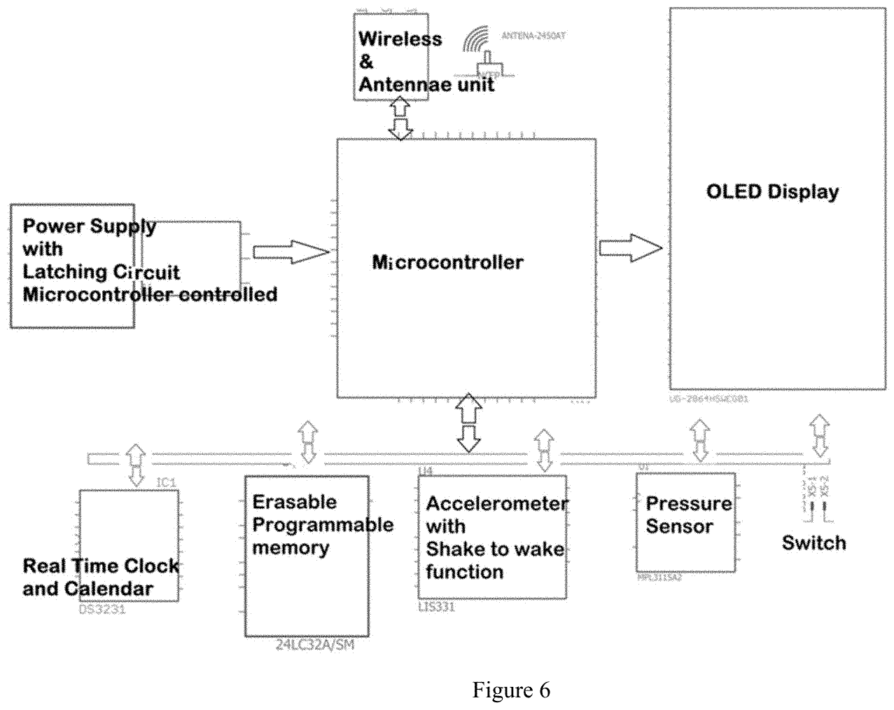

FIG. 6 depicts a computing-architecture within the inhaler 100 as depicted in FIG. 1. More specifically, the present FIG. 6 depicts a computing-system embedded within the inhaler 100, and comprises:

a) an assembly of power supply with a microcontroller enabled latch protection against over/under current,

b) a microprocessor/microcontroller to perform the necessary decision-making functions pertaining to the inhaler as illustrated in FIG. 1 to FIG. 5,

c) a real-time clock/calendar to maintain accurate time-keeping when main power to the inhaler 100 is interrupted

d) an EEPROM rendering a non-volatile memory source for storing data captured during the operation,

e) an accelerometer for sensing a shake imposed upon the inhaler and activating the inhaler from sleep/stand-by state,

f) a pressure-sensor (e.g. a piezoelectric sensor, a vacuum pressure-transducer) for sensing the inhalation/exhalation pressure etc;

g) an organic light-emitting diode (OLED) based display for rendering an output-display; and

h) a wireless antennae for wirelessly and bi-directionally communicating with a remotely-located transceiver device, such as a mobile-phone.

Overall, the inhaler 100 illustrated by the present invention is a programmable-electromechanical breath actuated inhaler that can be programmed to provide for efficacious delivery of the selected medication to a given patient/user. The invention provides a durable, battery-operated device (which can be recharged through USB cable) for administering aerosolized medication having a water tight disposable housing that contains the actuation mechanism. Moreover, the inhaler 100 stands constituted within a close water-proof housing, as compared to the conventional actuator body of the pMDI.

According to an embodiment, the inhaler 100 keeps the count of dose taken, gives real time feedback to patient if any critical step during the inhalation maneuver is missed e.g. shaking before inhalation, exhalation before inhalation start, incomplete inhalation etc., and displays the outcomes for each actuation on the device display. The inhaler 100 also achieves transmission of the data to the application with respect to each actuation, thereby giving a detailed report of spatial and temporal-usage summary with respect to each actuation by the user.

An electronic chip controller attached with on-board device memory located on the electronic printed circuit board renders sufficient storage of complete data for the number of doses taken as well as details of each actuations, thereby eliminating the need for the device to be in proximity to the smart-phone when dose is taken. A further advantage is that with a tracking module having own internal memory, inhaler and smart phone can occasionally sync without possibility of losing data.

While specific language has been used to describe the disclosure, any limitations arising on account of the same are not intended. As would be apparent to a person in the art, various working modifications may be made to the method in order to implement the inventive concept as taught herein.

It is understood that the above description is intended to be illustrative, and not restrictive. It is intended to cover all alternatives, modifications and equivalents as may be included within the spirit and scope of the invention as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein.

Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1. A breath-actuated inhaler, comprising:
a motion-generator configured to render motion upon actuation by a user;
a canister;
an arrangement configured to transform the generated motion to enable the canister to undergo translation-motion; and
a metering-valve operably connected to the canister configured to cause a metered-release of the canister's contents as a spray, based upon said translation-motion.

2. The inhaler as claimed in claim 1, wherein the motion-generator is an electronically-controllable rotary motion-source and configured to generate clock-wise and anti-clockwise motion and cause translation motion of the canister in either of the directions.

3. The inhaler as claimed in claim 1, wherein the arrangement to convert the generated motion into translation motion comprises:
an externally-threaded lead-screw connected to the motion generator; and
a screw-nut having internal-threads coupled to said lead-screw and adapted to undergo translation-motion along a screw-length upon the rotation of the lead-screw.

4. The inhaler as claimed in claim 3, further comprising a cap defined by:
a recessed top-portion acting as a screw-nut to thereby translate across the length of the lead-screw, and
a lower-portion configured to hold a portion of the canister to thereby enable the translation motion of the cap and the canister as a single unit.

5. The inhaler as claimed in claim 1, wherein the arrangement to convert the generated motion into translation-motion comprises a cam-arrangement linked to the motion-generator configured to convert the generated rotary-motion into translation-motion.

6. The inhaler as claimed in claim 1, further comprising:
a nozzle rigidly disposed within the lower-housing and connected to the metering-valve configured to cause a spray formation; and
a flow-director within the lower-housing configured to direct said spray outside the lower-housing.

7. The inhaler as claimed in claim 1, further comprising at least one of:
a position-sensor configured to sense the position of at least one of the motion-generator and the canister;
a motion-sensor to determine extent of motion underwent by the motion-generator and the canister; and
a pressure-sensor provided within a flow-path of the spray to monitor an inhalation-flow as generated corresponding to the spray.

8. The inhaler as claimed in claim 1, further comprising a data-logging and processing system configured to electronically determine at least one of:
a) at least one position parameter captured by a position sensor with respect to at least one of the motion-generator and the canister;
b) at least one motion-parameter captured by a motion-sensor with respect to at least one of motion-generator and the canister and
c) a pressure-parameter associated with the spray within the flow-path, wherein the pressure-parameter is captured by at-least one pressure-sensor.

9. The inhaler as claimed in claim 8, wherein the data-logging and processing-system is configured to cause one or more of:
a) control of the timing of actuation of canister;
b) control of the timing of an auto return mechanism of canister;
c) control of the time of venting of the metered dose inhaler;
d) real-time tracking of the inhalation flow-rate;
e) real-time control of amount of medication for inhalation;
f) derivation of an inhalation-profile of the user;
g) judgment of inhalation done by the user;
h) acknowledgement of a complete-dosage as undertaken by the patient;
i) maintaining a count of dosages in a given period of time;
j) sending a real-time feedback regarding an executed inhalation; and
k) communication of data pertaining to operation of the inhaler to a remote-device.

10. A method implemented in a breath-actuated inhaler, comprising:
converting a rotary motion from a motion generator into translation-motion upon a user-actuation to thereby depress a canister;
monitoring at least one of:
an inhalation-flow generated due to said user-actuation, through a pressure-sensor;
motion as underwent by the motion generator and the canister, through a motion-sensor; and
an instantaneous position exhibited by the motion-generator and the canister, through a position sensor; and
processing data obtained based on said monitoring to execute one or more:
a) control of the timing of actuation of the canister;
b) control of the timing of an auto return mechanism of the canister;
c) control of the time of venting of the metered dose inhaler;
d) real-time tracking of the inhalation flow-rate;
e) real-time control of amount of medication for inhalation;
f) derivation of an inhalation-profile of the user;
g) judgment of inhalation done by the user;
h) acknowledgement of a complete-dosage as undertaken by the patient;
i) maintaining a count of dosages in a given period of time;
j) sending a real-time feedback regarding an executed inhalation; and
k) communication of data pertaining to operation of the inhaler to a remote-device.
